(19) United States
(12) Patent Application Publication
(10) Pub. No.: US 2026/0048262 A1
(43) Pub. Date: Feb. 19, 2026

(54) NEUROMODULATION DEVICE ASSOCIATED WITH SLEEP CYCLE

(71) Applicant: Nu Eyne Co., LTD, Seoul (KR)

(72) Inventors: Do-Hyoung KIM, Seoul (KR); Joo-Wan SEO, Seoul (KR); Tae-Wan KIM, Gwangmyeong-si (KR); Young-Min PARK, Seoul (KR)

(21) Appl. No.: 18/804,168

(22) Filed: Aug. 14, 2024

(51) Int. Cl. A61N 1/36

(52) U.S. Cl. CPC A61N 1/36031; A61N 1/36034

(57) ABSTRACT

The present invention relates to a neuromodulation device associated with a sleep cycle, and relates to a neuromodulation device associated with a sleep cycle, which applies a therapeutic stimulation signal to an electrode module in association with sleep cycle and sleep stage signals generated for each user. To this end, disclosed is a neuromodulation device associated with a sleep cycle comprising: an impedance detection unit for detecting impedance to detect whether the neuromodulation device is attached to a skin; and a stimulation adjustment unit for transmitting a therapeutic stimulation signal to an electrode module in synchronization with a sleep cycle signal of each user generated on the basis of at least one sleep detection signal.

### BACKGROUND OF THE INVENTION

Field of the Invention The present invention relates to a neuromodulation device associated with a sleep cycle, and more specifically, to a neuromodulation device associated with a sleep cycle, which applies a therapeutic stimulation signal to an electrode module in association with sleep cycle and sleep stage signals generated for each user.

### Background of the Related Art

Conventional neuromodulation devices used to treat ADHD apply electrical stimulation by applying therapeutic waveforms with adjusted frequency and pulse width to electrode modules attached to the forehead of a user. Conventional electrical stimulators have a problem in that electrical stimulation according to a sleep cycle and tossing and turning of the user during sleep may not be adjusted.

### SUMMARY OF THE INVENTION

Therefore, the present invention has been made in view of the above problems, and it is an object of the present invention to provide an invention capable of enhancing the therapeutic effect by generating sleep cycle and sleep stage signals of the user and applying a therapeutic stimulation signal in synchronization with the generated sleep cycle and sleep stage signals.

However, the object of the present invention is not limited to the object mentioned above, and unmentioned other objects will be clearly understood by those skilled in the art from the following description.

To accomplish the above object, according to one aspect of the present invention, there is provided a neuromodulation device associated with a sleep cycle, the device comprising: an impedance detection unit for detecting impedance to detect whether the neuromodulation device is attached to a skin; and a stimulation adjustment unit for transmitting a therapeutic stimulation signal to an electrode module in synchronization with a sleep cycle signal of each user generated on the basis of at least one sleep detection signal.

In addition, the neuromodulation device further comprises a sleep detection sensor unit for generating at least one sleep detection signal, wherein the sleep detection sensor unit includes: an inertial measurement sensor unit for generating a first sleep detection signal of the user; and a photoplethysmogram sensor unit for generating a second sleep detection signal of the user.

In addition, the neuromodulation device further comprises: a sleep cycle estimation unit for generating a sleep cycle signal for estimating a sleep cycle of each user according to at least any one among the first sleep detection signal and the second sleep detection signal; and a stimulation adjustment synchronization unit for synchronizing the therapeutic stimulation signal generated by the stimulation adjustment unit based on the sleep cycle signal of each user.

In addition, the sleep cycle signal estimated by the sleep cycle estimation unit is a cycle signal in which a deep sleep stage where the user gradually enters a deep sleep from a wake stage where the user is awake is repeated in each stage, and the stimulation adjustment unit adjusts the stimulation intensity or stimulation time in synchronization with each sleep stage or sleep cycle.

In addition, the stimulation adjustment unit synchronously controls the therapeutic stimulation signal so that the stimulation intensity or stimulation time is changed in a downward stage and an upward stage of the sleep cycle.

In addition, the neuromodulation device further comprises a tossing and turning detection unit for generating a tossing and turning detection signal for detecting tossing and turning of the user during sleep on the basis of the first sleep detection signal.

In addition, the stimulation adjustment unit changes and adjusts the stimulation intensity or stimulation time according to the tossing and turning detection signal when synchronization of the therapeutic stimulation signal is controlled.

In addition, the stimulation adjustment unit synchronously controls the therapeutic stimulation signal by gradually increasing the stimulation intensity according to the sleep stage or sleep cycle on the basis of feedback of the tossing and turning detection signal when tossing and turning of the user is relatively low, and gradually decreasing the stimulation intensity according to the sleep stage or sleep cycle on the basis of the feedback of the tossing and turning detection signal when the tossing and turning is relatively high.

### BRIEF DESCRIPTION OF THE DRAWINGS

Since the following drawings attached to this specification illustrate preferred embodiments of the present invention and function to help further understanding of the technical spirit of the present invention, together with the detailed description of the present invention, the present invention should not be interpreted as being limited to the matters described in the drawings.

FIG. 1 is a view showing a schematic configuration of a neuromodulation device associated with a sleep cycle according to an embodiment of the present invention.

FIG. 2 is a view showing sleep stages and sleep cycles according to an embodiment of the present invention.

### DETAILED DESCRIPTION OF THE PREFERRED EMBODIMENT

Hereinafter, a preferred embodiment of the present invention will be described with reference to the drawings. In addition, the embodiment described below do not unduly limit the contents of the present invention described in the patent claims, and the entire configuration described in this embodiment cannot be said to be essential as a solution of the present invention. In addition, descriptions of matters that are obvious to those skilled in the art may be omitted, and descriptions of such omitted components (methods) and functions may be sufficiently referenced within the scope that does not deviate from the technical spirit of the present invention.

A neuromodulation device associated with a sleep cycle according to an embodiment of the present invention is a device that controls a therapeutic stimulation signal in synchronization with a sleep stage and a sleep cycle generated for each user. The neuromodulation device associated with a sleep cycle according to the present invention may treat autism, attention-deficit hyperactivity disorder (ADHD), or insomnia by applying a predefined signal waveform (e.g., frequency, duty ratio, and the like are predefined) to a human body, e.g., by being worn on the forehead. Hereinafter, the neuromodulation device (hereinafter, referred to as a neurostimulator or a stimulator) associated with a sleep cycle according to an embodiment of the present invention will be described in detail with reference to the attached drawings.

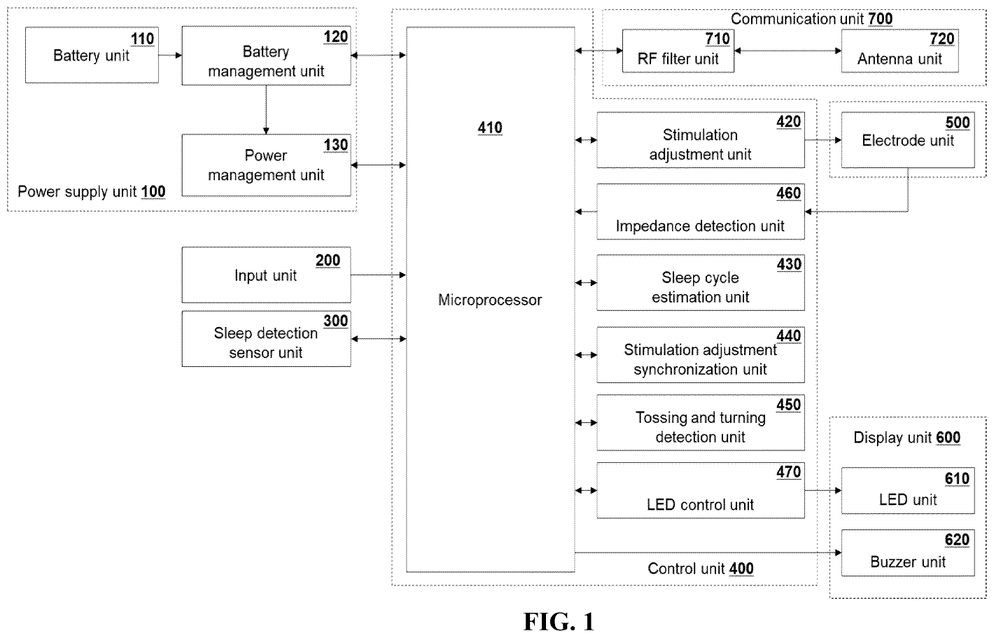

Referring to FIG. 1, a neuromodulation device associated with a sleep cycle (or period) according to an embodiment of the present invention roughly includes a power supply unit 100, an input unit 200, a sleep detection sensor unit 300, a control unit 400, an electrode unit 500, a display unit 600, and a communication unit 700.

The power supply unit 100 includes a rechargeable battery unit 110, a battery management unit 120 for managing the battery unit 110, and a power management unit 130 for managing entire power of the neuromodulation device. The battery management unit 120 and the power management unit 130 are connected to a microprocessor 410 described below.

The input unit 200 includes buttons for controlling the operation of the neuromodulation device or setting a stimulation signal. When the neuromodulation device is set up initially, the user may set a stimulation intensity and a stimulation time according to his/her condition on that day through the manual buttons. The stimulation intensity and the stimulation time initially set by the manual buttons are changed and adjusted by a stimulation adjustment unit 420 described below, and a therapeutic stimulation signal with the changed and adjusted stimulation intensity and stimulation time is output to the electrode unit 500. At this point, the signal output from the stimulation adjustment unit 420 is output in synchronization with a sleep cycle or a sleep stage.

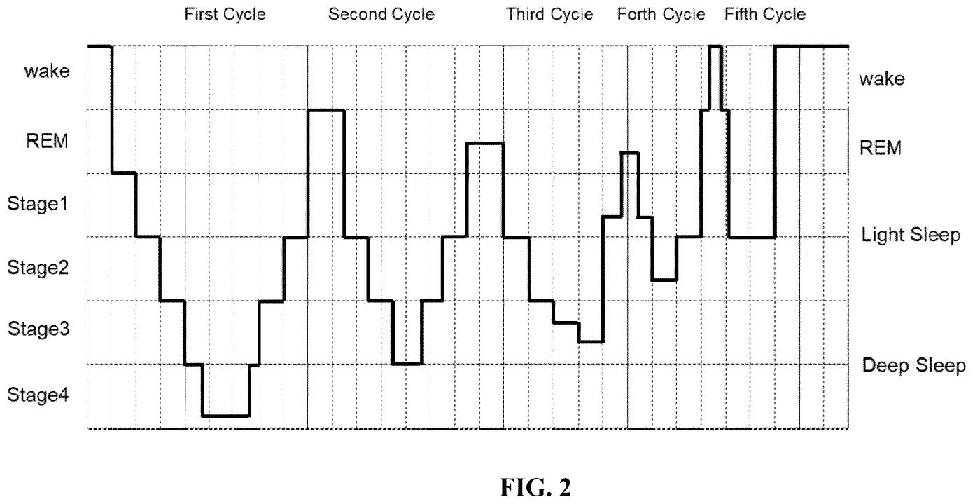

The sleep detection sensor unit 300 includes an inertial measurement (IMU) sensor and a photoplethysmogram (PPG) sensor, and generates a sleep detection signal detecting sleep of a user. The inertial measurement sensor generates a first sleep detection signal of the user, and the PPG sensor generates a second sleep detection signal of the user. The sleep cycle and sleep stage of the user may be estimated as shown in FIG. 2 through the detection signals of the inertial measurement sensor and the PPG sensor. At this point, the sleep cycle and sleep stage may be estimated using only the inertial measurement sensor, and preferably, the PPG sensor may be added as a supplement to the inertial measurement sensor to estimate the sleep cycle and sleep stage.

Functionally, the microprocessor 410 roughly includes a stimulation adjustment unit 420, a sleep cycle estimation unit 430, a stimulation adjustment synchronization unit 440, a tossing and turning detection unit 450, an impedance detection unit 460, and an LED control unit 470. The microprocessor 410 operates by a program stored in the RAM or ROM to control the neuromodulation device. The microprocessor 410 is interfaced with the power supply unit 100, the input unit 200, the sleep detection sensor unit 300, the display unit 600, and the communication unit 700, respectively.

The stimulation adjustment unit 420 supplies the generated therapeutic stimulation signal to the electrode unit 500. At this point, the therapeutic stimulation signal is output in synchronization with the sleep cycle or sleep stage as a signal in which the stimulation intensity and the stimulation time are adjusted in synchronization with the sleep cycle or sleep stage.

The sleep cycle estimation unit 430 estimates the sleep cycle and sleep stage for each user as shown in FIG. 2 using the first and second sleep detection signals input through the inertial measurement sensor and the PPG sensor.

As shown in FIG. 2, estimated sleep cycles of the user are generated as, for example, a first cycle, a second cycle, a third cycle, a fourth cycle, and a fifth cycle. Estimated sleep stages of the user are generated to be classified into a wake stage, a REM stage, Stage 1, Stage 2, Stage 3, and Stage 4.

The stimulation adjustment unit 420 outputs a therapeutic stimulation signal to the electrode unit 500 in synchronization with the estimated sleep cycle and sleep stage signals (hereinafter, collectively referred to as a sleep cycle signal) as shown in FIG. 2. For example, when sleep progresses from the wake stage to Stage 4 (deep sleep) (downward stage), the stimulation intensity may be adjusted to gradually increase. When the tossing and turning response of the user increases after adjusting the stimulation intensity at each stage, the stimulation intensity may be lowered back to the previous stage level. Meanwhile, when the sleep progresses from Stage 4 (deep sleep) to the wake stage (upward stage), the stimulation intensity may be adjusted to gradually decrease. Even at this point, the stimulation intensity is adjusted after observing the tossing and turning response of the user. At this point, the stimulation time, as well as the stimulation intensity, may also be controlled in synchronization with the sleep cycle and sleep stage. The stimulation time may be a time of applying a therapeutic stimulation signal or may be the pulse width of the therapeutic stimulation signal.

The stimulation adjustment synchronization unit 440 generates a stimulation synchronization signal for synchronizing the therapeutic stimulation signal on the basis of the sleep cycle and sleep stage signals estimated by the sleep cycle estimation unit 430. The stimulation synchronization signal may be generated at each sleep stage or sleep cycle. The stimulation adjustment unit 420 outputs a therapeutic stimulation signal to the electrode unit 500 in synchronization with the stimulation synchronization signal transmitted from the stimulation adjustment synchronization unit 440.

The tossing and turning detection unit 450 detects tossing and turning while the user sleeps on the basis of the first sleep detection signal transmitted from the inertial measurement sensor and generates a tossing and turning detection signal. The tossing and turning detection unit 450 transmits the generated tossing and turning detection signal to the stimulation adjustment unit 420. The stimulation adjustment unit 420 changes and adjusts the stimulation intensity and the stimulation time at each sleep stage or sleep cycle according to the input tossing and turning detection signal.

The impedance detection unit 460 detects whether the user has properly attached the neuromodulation device through impedance measurement.

The LED control unit 470 controls an LED unit 610 and a buzzer unit 620 of the display unit 600 so that the user may easily confirm the state of the neuromodulation device.

According to the present invention as described above, the therapeutic effect can be enhanced by generating sleep cycle and sleep stage signals of the user and applying a therapeutic stimulation signal in synchronization with the generated sleep cycle and sleep stage signals.

In describing the present invention, descriptions of matters that are obvious to those skilled in the art may be omitted, and descriptions of the omitted components (methods) and functions may be sufficiently referenced within the scope that does not deviate from the technical spirit of the present invention. In addition, the components of the present invention described above have been described only for convenience of explanation of the present invention, and components not described herein may be added within the scope that does not deviate from the technical spirit of the present invention.

The description about the configuration and function of each part described above has been provided separately only for convenience of explanation, and any one configuration and function may be implemented to be integrated into another component or may be implemented to be further subdivided as needed.

Although it is described above with reference to an embodiment of the present invention, the present invention is not limited thereto, and various modifications and applications are possible. That is, those skilled in the art will easily understand that many modifications are possible without departing from the spirit of the present invention. In addition, it should be noted that when it is determined that a specific description of a known function related to the present invention and its configuration or a combination relationship for each configuration of the present invention may unnecessarily obscure the gist of the present invention, the specific description is omitted.

### DESCRIPTION OF SYMBOLS

100: Power supply unit
110: Battery unit
120: Battery management unit
130: Power management unit
200: Input unit (button unit)
300: Sleep detection sensor unit
400: Control unit
410: Microprocessor
420: Stimulation adjustment unit
430: Sleep cycle estimation unit
440: Stimulation adjustment synchronization unit
450: Tossing and turning detection unit
460: Impedance detection unit
470: LED control unit
500: Electrode unit
600: Display unit
610: LED unit
620: Buzzer unit
700: Communication unit
710: RF filter unit
720: Antenna unit

What is claimed is:

1. A neuromodulation device associated with a sleep cycle, the device comprising:
an impedance detection unit for detecting impedance to detect whether the neuromodulation device is attached to a skin; and
a stimulation adjustment unit for transmitting a therapeutic stimulation signal to an electrode module in synchronization with a sleep cycle signal of each user generated on the basis of at least one sleep detection signal.

2. The neuromodulation device according to claim 1, further comprising a sleep detection sensor unit for generating at least one sleep detection signal, wherein the sleep detection sensor unit includes:
an inertial measurement sensor unit for generating a first sleep detection signal of the user; and
a photoplethysmogram sensor unit for generating a second sleep detection signal of the user.

3. The neuromodulation device according to claim 2, further comprising:
a sleep cycle estimation unit for generating a sleep cycle signal for estimating a sleep cycle of each user according to at least any one among the first sleep detection signal and the second sleep detection signal; and
a stimulation adjustment synchronization unit for synchronizing the therapeutic stimulation signal generated by the stimulation adjustment unit based on the sleep cycle signal of each user.

4. The neuromodulation device according to claim 3, wherein the sleep cycle signal estimated by the sleep cycle estimation unit is a cycle signal in which a deep sleep stage where the user gradually enters a deep sleep from a wake stage where the user is awake is repeated in each stage, and the stimulation adjustment unit adjusts the stimulation intensity or stimulation time in synchronization with each sleep stage or sleep cycle.

5. The neuromodulation device according to claim 4, wherein the stimulation adjustment unit synchronously controls the therapeutic stimulation signal so that the stimulation intensity or stimulation time is changed in a downward stage and an upward stage of the sleep cycle.

6. The neuromodulation device according to claim 5, further comprising a tossing and turning detection unit for generating a tossing and turning detection signal for detecting tossing and turning of the user during sleep on the basis of the first sleep detection signal.

7. The neuromodulation device according to claim 6, wherein the stimulation adjustment unit changes and adjusts the stimulation intensity or stimulation time according to the tossing and turning detection signal when synchronization of the therapeutic stimulation signal is controlled.

8. The neuromodulation device according to claim 7, wherein the stimulation adjustment unit synchronously controls the therapeutic stimulation signal by gradually increasing the stimulation intensity according to the sleep stage or sleep cycle on the basis of feedback of the tossing and turning detection signal when tossing and turning of the user is relatively low, and gradually decreasing the stimulation intensity according to the sleep stage or sleep cycle on the basis of the feedback of the tossing and turning detection signal when the tossing and turning is relatively high.
